(19) United States
(12) Patent Application Publication
(10) Pub. No.: US 2011/0040282 A1
(43) Pub. Date: Feb. 17, 2011

(54) Controllably Stiffenable Tube

(75) Inventor: Bernhard Uihlein, Dettingen (DE)

Correspondence Address: CROWELL & MORING LLP;INTELLECTUAL PROPERTY GROUP, P.O. BOX 14300, WASHINGTON, DC, 20044-4300, US

(73) Assignee: EPFLEX FEINWERKTECHNIK GMBH, Dettingen/Ems (DE)

(21) Appl. No.: 12/279,502

(22) PCT Filed: Feb. 14, 2007
(86) PCT No.: PCT/EP07/01258

§ 371 Date: Sep. 10, 2010

(30) Foreign Application Priority Data
Feb. 15, 2006 (DE) 10 2006 007 974.4
Apr. 19, 2006 (DE) 10 2006 018 489.0

(51) Int. Cl. A61M 25/00

(52) U.S. Cl. 604/525

(57) ABSTRACT

A tube that can be stiffened in a controlled manner has a pressure channel for controllable production of a stiffening overpressure by introduction of a pressure medium. The tube has several flexible stiffening elements, of which at least one is stable with respect to tensile force and which are pressed onto one another when acted upon by the stiffening overpressure with the result that the tube is changed from a more flexible state to a more flexurally stiff state. A channel is separate from the pressure channel and, in the stiffened state of the tube, forms a working channel whose course corresponds to the tube shape.

### BACKGROUND AND SUMMARY OF THE INVENTION

The present invention relates to a tube that can be stiffened in a controlled manner, usable particularly as a catheter in the medical field and the like.

Conventional catheters used in the medical field are relatively stiff because a corresponding stiffness is required in the operating or working position in which the catheter is pushed into a tissue canal of a human or animal body, such as a vein or artery. For inserting such a conventional catheter, a guide wire is therefore normally first introduced into the tissue canal, along which the catheter is then pushed following the guide wire.

For the foregoing purpose, WO 2004/035124 A1 suggests guide wires which can be stiffened in a controlled manner and which have several strands of wire that can be acted upon by magnetic attraction forces by generating magnetic fields in order to change the guide wire into a flexurally stiff condition. By deactivating the magnetic fields, the guide wire is returned to its more flexible condition, as required, aided the by the introduction of pressure into gaps between the strands of wire.

In another embodiment of that published document, a central, flexible, radially expandable tube membrane is surrounded by several strands of wire arranged in a distributed manner in the circumferential direction. The strands of wire, in turn, are surrounded by an outer cover with a cast-in helical wire. By the admission of an overpressure, the tube membrane exerts radially outward pointing pressure forces which press the strands of wire against the outer cover and thereby stiffen the guide wire. In WO 91/05507 A1, an insertion device for tube-shaped fiber-optical instruments, especially colonoscopes, has a ring-shaped underpressure channel that is bounded toward the outside by an outer, flexible but barely expandable tube jacket and, toward the inside, by an inner expandable tube jacket. The inner tube jacket is simultaneously used as the boundary for a central working channel used, for example, for rinsing and suction operations. When evacuating the underpressure channel, the inner tube jacket is supported against the outer tube jacket, preferably by suitable supporting bodies arranged on the exterior side of the inner tube jacket and/or on the interior side of the outer tube jacket, whereby the introduction device is stiffened. As a result of this pressure compensation, that is, release of the underpressure, the connection of the inner tube jacket with the outer tube jacket is released again and the introduction device is again more flexible.

An endoscope also based on the immediately above-mentioned functioning principle is disclosed in U.S. Pat. No. 4,815,450. The underpressure ring channel used there has an inner and a coaxial outer part which are separated by a wall through which a fluid connection exists between the two channel parts and by which the loose pellets in the inner part, as supporting bodies, are kept separate from the outer part.

WO 2005/042078 A1 discloses a device, especially a lock or a catheter, for the at least partial insertion into a body canal, having an oblong, outer enveloping body, an oblong inner body surrounded by the latter on the circumference side at least in sections and a control device formed by the arrangement and construction of the enveloping body and of the inner body itself. The control device is permits a relative movement between the enveloping body and the inner body such that it can be controlled in a targeted manner or at least making this relative movement more difficult. The control device particularly is constructed such that the friction between the enveloping body and the inner body can be controlled mechanically and/or by a pressure or a vacuum, electric polarization, magnetization and/or by a molecular alteration. The material of the enveloping body and the inner body preferably has a flexible but distortion-proof construction.

In one known embodiment, a pressure medium such as compressed air can be introduced by the above-mentioned control device preferably in/at the space between the enveloping body and the inner body, or a vacuum can be applied. In the latter case, the enveloping body and/or the inner body are made of an expandable, that is, not dimensionally stable material. By evacuating the space between the enveloping or the inner body or generating overpressure in the interior of the inner body, the inner body, while radially expanding, comes to rest flatly against the interior wall of the enveloping body, so that the friction between the enveloping and the inner body is increased. As a result, a stiffening of the entire device is to be caused. In order to sufficiently ensure this for the enveloping and inner bodies implemented as tubes, in corresponding embodiments, a polygonal cross-section and a relative rotatability between the enveloping and the inner body or the generating of attracting magnetic fields is preferably provided.

An object of the present invention is to provide a tube that can be stiffened in an improved controlled or targeted manner, and be returned to its less stiff condition.

The present invention has achieved this object by providing a tube which can be stiffened in a controlled manner and has a pressure channel for controllable generation of a stiffening overpressure by introducing therein a pressure medium, a plurality of flexible stiffening elements of which at least one is stable with respect to tensile force and which are pressed onto one another when acted upon by the stiffening overpressure to stiffen the tube from a more flexible to a flexurally stiffer state, and a channel which is separated from the pressure channel and which, in the stiffer state of the tube, forms a working channel corresponding to a shape of the tube.

The tube is configured such that, as a result of the corresponding introduction of a pressure medium into its pressure channel, an overpressure is generated in a controlled manner, by which by which the tube is changed to a flexurally stiffer state. For this purpose, the tube comprises flexible stiffening elements of which at least one is stable with respect to tensile force and which rest against one another such that their tube shape is changed under the effect of the pressure forces generated by the overpressure. When relieved from pressure, that is, when the overpressure is released, the tube resumes its more flexible state.

A usable channel separated from the pressure channel will also be maintained preferably with a cross-section essentially unchanged in comparison with the pressure-relieved tube state when the tube is changed to the flexurally stiffer state, which normally represents the functional state of the tube when in use, for example, as a catheter. Because of the separation from the pressure channel, the usable channel can additionally be configured specifically for given usage requirements independently of the pressure medium in the pressure channel.

The tube configuration according to the present invention such that it is changed to its flexurally stiffer state by overpressure and not by underpressure has, among others, advantages with respect to a greater freedom of choice for the shape and the material of the flexible stiffening elements and the handling of the tube when in use. In addition, the equipment expenditures for providing overpressure are generally lower than for providing an underpressure resulting in comparably high pressure forces. Furthermore, an admission of overpressure normally can be caused with clearly shorter reaction times than an admission of underpressure.

An advantage for achieving a reliable stiffening state is obtained by configuring at least one of the flexible stiffening elements and preferably at least two flexible stiffening elements, which interact in a frictionally engaged and/or interlocking manner, to be stable with respect to tensile force. Thereby, in the state when the tube shape is stiffened, tensile forces can be absorbed; that is, those forces can be withstood in a dimensionally stable manner which can act mainly in the longitudinal direction. Such tensile forces may, for example be caused in that external flexural loads act upon the stiffened tube which result in corresponding static friction forces or interlocking forces upon the flexible stiffening elements. This may further result in such tensile forces that the tube is stiffened in a position which represents no stable position in the force-free unstiffened state, so that the tube attempts to move from its stiffened position into its initial position which results in corresponding force loads for one or more of the flexible stiffening elements.

As a further development of the invention, the flexible stiffening elements are equipped for a frictionally engaged and/or form-locking stiffening interaction, and/or they comprise a flexible sleeve and at least one flexible longitudinal rod-type element, and/or they comprise a flexible outer sleeve in the form of an outer stiffening tube part and an inner stiffening tube part surrounded by the latter which, under the effect of the stiffening overpressure, is pressed against the interior side of the outer stiffening tube part so as to stiffen the tube shape. In the latter case, the two stiffening tube parts may, for example, consists of a wire mesh material, whereby they become sufficiently stable to tensile force when, during the admission of pressure, the inner stiffening tube part is pressed in a frictionally engaged and/or form-locking manner against the interior side of the outer stiffening tube part in order to establish the flexurally stiffer tube condition.

In the further development of the invention, the pressure channel is constructed as a ring channel which is bounded by an inner and an outer tube membrane, at least the outer tube membrane being flexible and radially expandable and as a result exerts an outward-pointing pressure force component on the adjacent flexible stiffening elements.

As a further development of the invention, the usable channel is formed by a flexible inner sleeve which is surrounded by the inner tube membrane or formed by the latter. Thereby, a simple manner, a material-related uncoupling of the exterior-side boundary of the usable channel by the flexible inner sleeve from the interior-side boundary of the pressure channel by the inner tube membrane is permitted. In other words, in this manner, the exterior wall of the usable channel provided by the flexible inner sleeve can be configured specifically for requirements arising for the usable channel as the working channel when the tube is in use, while the inner tube membrane can primarily be designed for the sealing function for the pressure medium. When the respective requirements meet one another, as an alternative, the inner tube membrane can simultaneously act as the exterior-side boundary of the usable channel.

As a further development of the invention, the flexible stiffening elements comprise a flexible outer sleeve and at least one inner member arranged between the outer tube membrane and the outer sleeve. The inner member is pressed against the outer sleeve under the effect of the radially outward-pointing pressure force component, in order to thereby cause the stiffening tube state, for example, by a frictional engagement and/or form-locking with the outer sleeve.

As a further development of the invention, the flexible outer sleeve and/or the flexible inner sleeve are formed by a tube jacket with a cast-in helical spring or a cast-in tubular woven fabric or by a helical spring. These implementations are advantageous with respect to manufacturing and function and can be implemented with the desired flexibility, that is, bendability.

As a further development, the at least one inner member is prefabricated together with the outer tube membrane on its exterior side, or together with the outer sleeve on its interior side. If several inner members are present, in each case, a portion of them can be prefabricated on the outer hose membrane or on the outer sleeve.

As a further development of the invention, several inner members are provided which, extending with an axial direction component and distributed in the circumferential direction, are arranged between the outer tube membrane and the outer sleeve.

As a further development of the invention, the longitudinal rod-type element(s) or inner member(s), which form at least a portion of the flexible stiffening elements, are those made of a flat-wire or round-wire material or of a stranded material or a rope material. The at least one inner member may also be implemented as a tubular braiding which can be radially expanded to the required extent and is stable with respect to longitudinal force. Corresponding flat-wires or round-wires, strands, or ropes or tubular braidings, which operate as flexible longitudinal rod-type elements or inner members, may extend particularly along the entire or at least the predominant length of the tube in the axial direction and, in the process, absorb tensile forces and, in this manner, stabilize the longitudinal course of the outer sleeve and thus of the tube as a whole also in curved sections in a stiffening fashion.

As a further development of the invention, the inner stiffening tube part is prefabricated together with the outer tube membrane on its exterior side or together with the outer stiffening tube parts on its interior side.

### BRIEF DESCRIPTION OF THE DRAWINGS

Other objects, advantages and novel features of the present invention will become apparent from the following detailed description of one or more preferred embodiments when considered in conjunction with the accompanying drawings.

FIG. 1 is a partial longitudinal sectional view of a tube that can be stiffened and can be used as a catheter, in a linear tube section in accordance with the present invention;

FIG. 2 is a cross-sectional view along line II-II of FIG. 1;

FIG. 3 is a view corresponding to FIG. 1 but in the stiffened in-use state of the tube;

FIG. 4 is a cross-sectional view along line IV-IV of FIG. 3;

FIG. 5 is a view corresponding to FIG. 3 of the stiffened tube but in a bent tube section;

FIG. 6 is a longitudinal sectional view corresponding to FIG. 3 but for a variation of the embodiment illustrated there;

FIG. 7 is a cross-sectional view along line VII-VII of FIG. 6;

FIG. 8 is a longitudinal sectional view corresponding to FIG. 3 but for another variation of the embodiment illustrated there;

FIG. 9 is a cross-sectional view along line IX-IX of FIG. 8;

FIG. 10 is a partial half-sectional longitudinal view of another variation of a tube that can be stiffened according to the present invention and used as a catheter, in a linear tube section; and

FIG. 11 is a cross-sectional view along line XI-XI of FIG. 10.

### DETAILED DESCRIPTION OF THE PREFERRED EMBODIMENTS

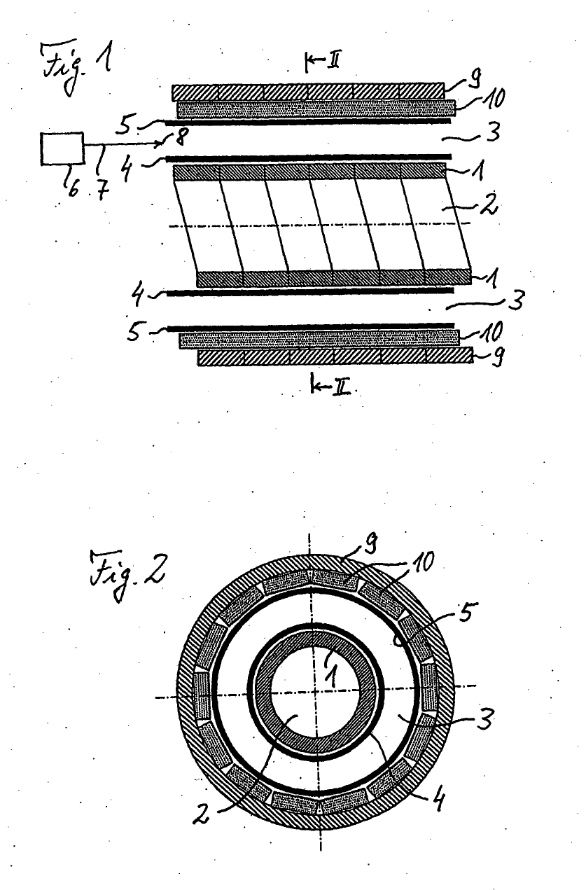

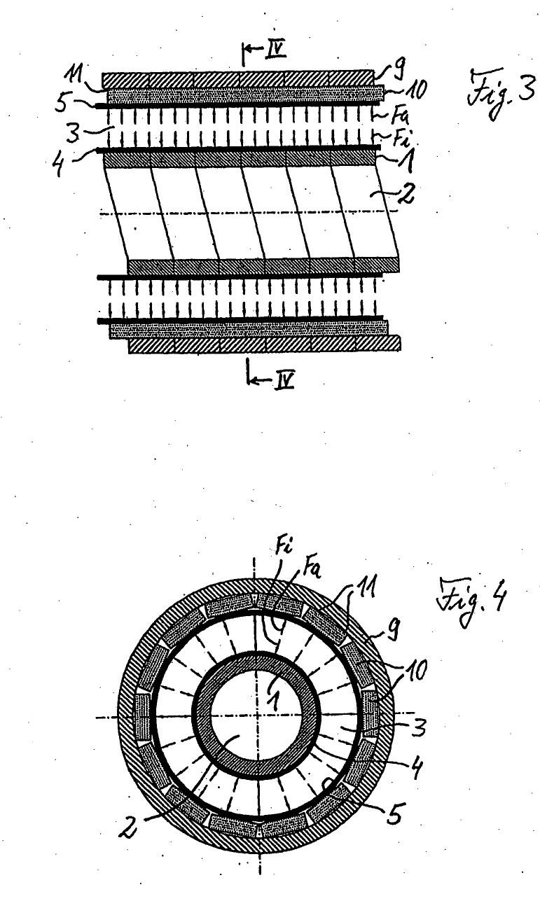

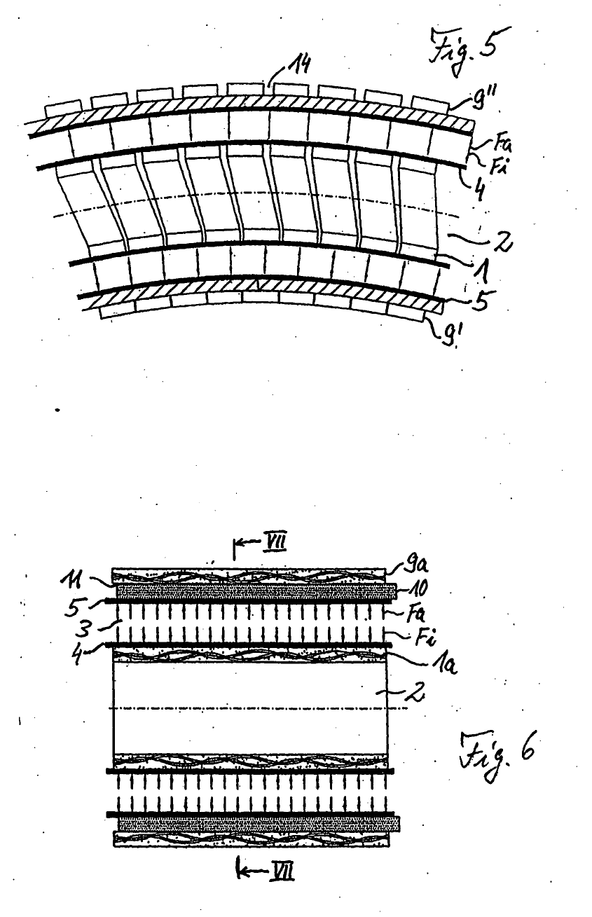

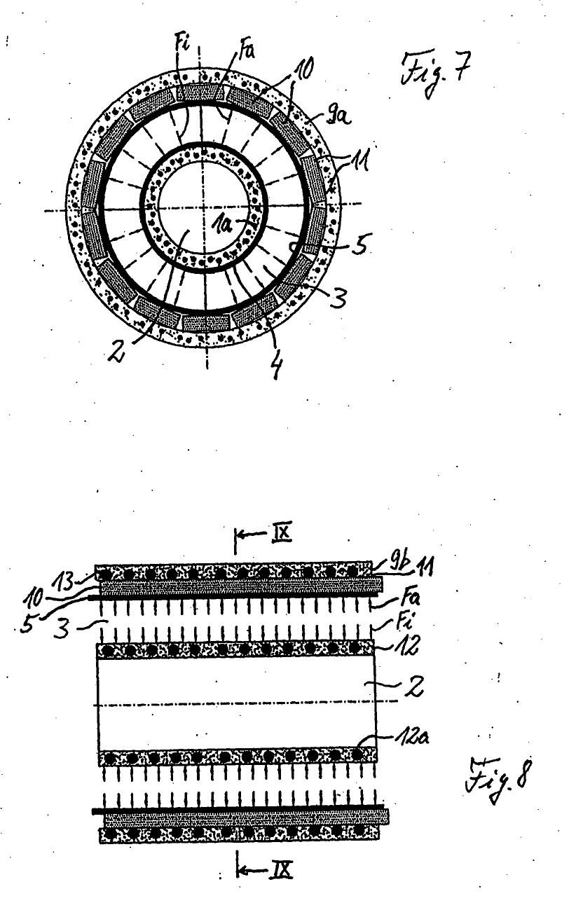

FIGS. 1 to 5 illustrate a first embodiment of a tube that can be stiffened, FIGS. 1 and 2 relating to a pressure-relieved, more flexible initial state of the tube, and FIGS. 3 to 5 relating to a pressure-loaded stiffened state of the tube. FIGS. 1 and 3 each are longitudinal sectional views of a linear axial portion of the tube; FIGS. 2 and 4 each being cross-sectional views of the tube; and FIG. 5 being a longitudinal sectional view of a curved axial portion of the tube. The dimensions of all illustrated elements of the tube are not necessarily true to scale but are each displayed for sufficiently clearly indicating the significance of these elements for the invention. This also applies to the additional hose variations illustrated in FIGS. 6 to 11.

All illustrated embodiments can be used, for example, as catheters for medical applications. Furthermore, tubes according to the invention that can be stiffened can also be used for other medical and non-medical purposes wherever there is a demand for a tube that can be changed in its bending stiffness in a variably controlled fashion; that is, from a more flexible initial state to a flexurally stiffer, stiffened state and can be returned to the more flexible initial state. In this case, depending on the requirement and application, the stiffened state may comprise several flexurally stiffer states with various increased bending stiffness which the tube can optionally be caused to assume, or only a single flexurally stiffer state is provided which has a defined increased bending stiffness.

The tube illustrated in FIGS. 1 to 5 comprises a central helical spring 1 as an inner flexible element which defines a central usable or working channel 2 in its interior. The inner helical spring 1 with the central usable channel 2 is coaxially surrounded by a ring-shaped pressure channel 3 which is in each case bounded in a fluid-tight or gas-tight manner radially toward the interior by an inner tube membrane 4 and radially toward the exterior by an outer tube membrane 5. A conventional pressure generating device 6, which is therefore shown only schematically in FIG. 1, along with a suitable connection 7 are provided to feed a pressure medium 8 such as compressed air or another gas or a liquid pressure medium, into the pressure channel in a controlled or regulated manner. In a manner that is not shown, for example, a suitable ring nozzle may be arranged as the connection element on the face side at the pressure channel 3.

The outer tube membrane 5 is surrounded by a flexible stiffening element arrangement comprising an outer sleeve in the form of another outer helical spring 9 and a plurality of inner members 10 which are inserted in the form of axially extending longitudinal rods in a uniformly distributed manner in the circumferential direction in an annular gap of an appropriate width between the outer tube membrane 5 and the outer helical spring 9. In the illustrated embodiment, there are fourteen longitudinal rods 10 arranged at a narrow distance side-by-side with an essentially rectangular cross-section, The rods 10 may be implemented particularly by corresponding flat wire material of metal or plastic. It is essential that the longitudinal rods 10 represent longitudinally stable, inner members which are stable with respect to tensile force and which are therefore capable of absorbing tensile forces acting in the longitudinal direction.

The inner and the outer tube membranes 4, 5 are manufactured of an expandable material. When therefore an overpressure is generated in the pressure channel 3 by feeding the pressure medium 8, that is, in the pressure-loaded tube condition of FIGS. 3 and 4, as a result of the effect of a radially outward-pointing pressure force component Fa, while expanding, the outer tube membrane 5 presses the adjacent longitudinal wire elements 10 radially toward the outside. As a result, the longitudinal wires or inner members 10, which previously had been disposed only loosely between the outer tube membrane 5 and the outer helical spring 9, are firmly pressed under pressure against the interior side of the virtually not expanding outer helical spring 9. Thereby, a frictionally engaged connection is established between the individual, axially extending inner members 10 and the outer helical spring 9. For this purpose, an overpressure in the range of approximately 5 bar to approximately 40 bar can, for example, be used.

As a result, the tube is changed to the flexurally stiffer, stiffened state. The reason is that the frictional engagement between the respective inner member 10 and the helical spring 9, together with the longitudinally stable nature of the inner members 10, prevents the helical spring 9 and thereby the tube as a whole from carrying out a bending motion from the thus fixed position. This is the result of the fact that such a bending motion by way of the frictional engagement would cause a tensile or compression deformation of the inner members in the longitudinal direction which these, however, do not permit because of their longitudinal stability. On the contrary, they absorb corresponding tensile or pressure loads in the longitudinal direction.

In embodiments with several flexurally stiffer tube conditions, a selection can be made thereamong, for example, by overpressures of different intensities. In the latter case, also the frictional contact surface between the flexible stiffening elements causing the stiffening is preferably appropriately coordinated with the providing of several flexurally stiffer conditions.

Specifically, the frictionally engaged connection is a result of the contact of each of the longitudinal wires 10 with the interior side of the outer helical spring 9. Depending on the construction of the helical spring 9 and of the longitudinal wires 10, the contact may be flat, linear or punctiform. In the example of FIGS. 1 to 4, primarily a line contact 11 of the two radially outer longitudinal side regions of each longitudinal wire 10 exists with the outer helical spring 9. As required, in addition or as an alternative to the frictionally engaged connection, a form-locking connection may be provided, for the purpose of which the contact surfaces of the outer sleeve 9 and of the inner members 10 which are pressed against one another are then provided with suitable corresponding surface profilings.

As a result of the frictionally engaged and/or form-locking connection, the inner members 10 that are stable with respect to tensile force or longitudinally stable stabilize the outer sleeve 9 in its longitudinal course also in possibly curved sections. FIG. 5 illustrates the tube stiffened by overpressure in such a curved section. While the outer helical spring 9 acting as the outer sleeve remains closed at the interior side 9′ of its curvature—i.e., its turns follow one another in an axially continuous manner there, as in linear tube sections, it opens up slightly on the exterior side 9″ of its curvature—i.e., its individual turns axially slightly move away from one another while forming corresponding gaps 14. The inner members 10 bridge these gaps and, because of their frictional engagement/form locking with the turns of the outer helical spring 9, ensure that the individual turns of the outer helical spring 9 are also held on the exterior side 9″ of the curvature of the helical spring 9 in a stable manner in their position which there is mutually spaced axially.

The inner members 10 thereby stabilize the longitudinal course of the outer helical spring 9 in the overpressure state also along such curved tube sections; that is, the tube as a whole reliably assumes its flexurally stiffer state also in these curved sections. The frictional engagement/form locking and the stability with respect to tensile force/longitudinal stability of the inner members 10 prevent that, in the flexurally stiffer state, the tube moves back into its not curved initial position or any other initial position which it assumes in the flexurally softer state.

As a result of the overpressure in the pressure channel 3, the inner tube membrane 4, in addition, is pressed radially toward the interior against the usable-channel-forming helical spring 1 with a radially inward-pointing pressure force component Fi, which thereby places itself closely against the latter from the outside. In this manner, the inner tube membrane 4 acts not only as a fluid-tight separation of the ring-shaped pressure channel 3 from the central working channel 2 but additionally protects the inner helical spring 1 reliably from any radial expansion in cases in which, while the tube is being used, a certain overpressure occurs in the working channel 2, and supports it in its longitudinal course also in curved sections according to FIG. 5.

When, starting from the stiffened tube condition of FIGS. 3 to 5, the pressure channel 3 is relieved again from the overpressure. That is, the pressure medium is drained from the pressure channel 3 until the pressure is equalized, the tube returns to its more flexible initial position of FIGS. 1 and 2, in which the inner tube membrane 4 loosely surrounds the inner helical spring 1, and the longitudinal wires 10 are situated without pressure in the annular gap between the outer tube membrane 5 and the outer helical spring 9.

In the example where the tube is used as a catheter, the catheter tube is inserted in its pressure-relieved, more flexible initial state of FIGS. 1 and 2 into a tissue canal of a human or animal body. This can take place without any problem because of the relatively slight bending stiffness. When the catheter tube is inserted, the overpressure is generated in the pressure channel 3, whereby the tube is changed to its flexurally stiffer, stiffened state while maintaining and stabilizing its longitudinal course resulting from the insertion into the tissue canal with generally linear and curved sections. In this state, the working operations will then be carried out for whose purpose the catheter tube was placed, for which the working channel 2 can be used which is present unchanged also in the stiffened tube state; for example, for introducing functional instruments, functional objects, liquid or gaseous media from the outside into the tissue canal and/or remove tissue particles, body fluids or the like from the tissue canal. After the conclusion of this work, the pressure channel 3 is relieved from pressure, so that the catheter tube resumes its more flexible initial condition and can be pulled out of the tissue canal without any problem.

FIGS. 6 and 7, 8 and 9 and 10 and 11, respectively illustrate variations of the tube that can be stiffened in a controlled manner and is shown in FIGS. 1 to 5, in each case, in the pressure relieved state corresponding to FIGS. 3 and 4. For an easier understanding the same reference symbols are used for identical or functionally equivalent components in the different embodiments.

The embodiment of FIGS. 6 and 7 differs from that of FIGS. 1 to 5 in that, instead of the inner helical spring 1, an inner tubular woven fabric element 1a is used as the flexible element bounding the central usable channel 2, and, instead of the outer helical spring 9, an outer tubular woven fabric element 9a is used as the flexible outer sleeve. The two tubular woven fabric elements 1a, 9a consist of a conventional casting of a tubular woven fabric, for example, with cast-in longitudinal threads or longitudinal wires, and are constructed such that they virtually do not expand in the radial direction. This has the result that, for the tube modified in this manner, they carry out the same functions as explained above with respect to the inner and outer helical spring 1, 9, to which reference can be made. Otherwise, the tube variation of FIGS. 6 and 7 also completely corresponds to that of FIGS. 1 to 4, so that for explaining the characteristics currently of interest and the resulting advantages, reference can also be made to the above explanations concerning the embodiment of FIGS. 1 to 4. Optionally, the inner tube membrane can be eliminated in this embodiment and its sealing function can be taken over by the inner tubular woven fabric element 1a alone.

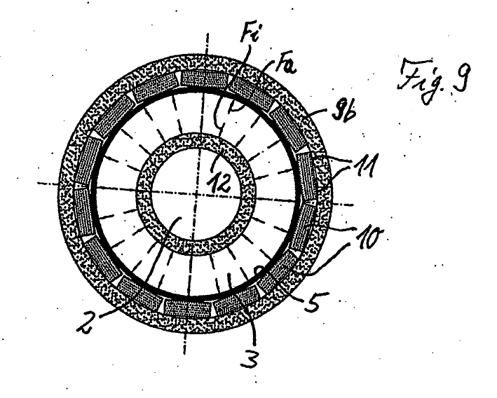

In the case of the tube variation of FIGS. 8 and 9, an inner tube jacket 12 with a cast-in helical spring 12a is used as a flexible inner element defining the usable channel 2. This inner tube jacket 12 combines the functions of the inner coil spring 1 and of the inner tube membrane 4 of the embodiment of FIGS. 1 to 5 and of the inner tubular woven fabric element 1a and of the inner tube membrane 4 of the embodiment of FIGS. 6 and 7 respectively. The reason is that, because of the embedding material surrounding the cast-in helical screw 12a, the inner tube jacket 12 provides the required fluid-tight separation of the central usable channel 2 from the pressure ring channel 3 surrounding it, and the cast-in helical spring 12a provides a sufficient stiffness, so that the inner tube jacket 12 is not noticeably compressed also under the effect of the radially inward-directed pressure forces Fi. As a result, the working channel 2 of the tube of FIGS. 8 and 9, also in the stiffened state, is again maintained to be interconnected and with an essentially unchanged cross-section.

Furthermore, in the case of the tube of FIGS. 8 and 9, an outer tube jacket 9b with a cast-in helical spring 13 acts as a flexile outer sleeve instead of the outer helical spring 9 of the embodiment of FIGS. 1 to 5 or of the outer tubular woven fabric element 9a of the embodiment of FIGS. 6 and 7. The effect and the method of operation of the outer tube jacket 9b correspond to those explained above with respect to the outer helical spring 9 of the embodiment of FIGS. 1 to 5, to which reference can be made.

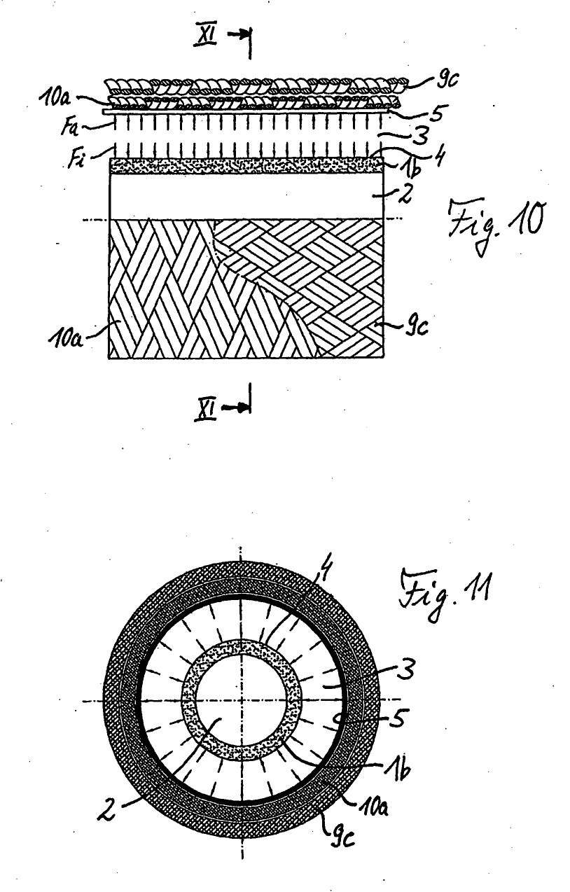

FIGS. 10 and 11 illustrate another tube variation in views similar to those of FIGS. 3 and 4. FIG. 10 shows a linear axial portion of the tube in a half-sectional longitudinal view; FIG. 11 is a cross-sectional view of the tube. The tube illustrated in FIGS. 10 and 11 comprises an inner flexible element 1b, for example, in the form of a central helical spring which defines a central usable or working channel 2 in its interior. The inner element 1b with the central usable channel 2 is coaxially surrounded by a ring-shaped pressure channel 3 which is in each case bounded in a fluid-tight or gas-tight manner radially toward the interior by the inner element 1b itself or by an inner tube membrane 4 surrounding this element 1b and is bounded radially toward the outside by an outer tube membrane 5. By way of a conventional pressure generating device, a pressure medium, such as compressed air or another gas or a liquid pressure medium, can be fed by way of a suitable connection into the pressure channel 3 in a controlled or regulated manner, as explained above. For example, a suitable ring nozzle (not shown) may be arranged as the connecting element, in a generally known manner, on the face side at the pressure channel.

The outer tube membrane 5 is surrounded by a flexible stiffening element arrangement which comprises a flexible outer sleeve in the form of an outer stiffening tube part 9c made of a wire mesh material and stable with respect to tensile force and a flexible inner stiffening tube part 10a also made of a wire mesh material stable with respect to tensile force which is inserted in an annular gap of an appropriate width between the outer tube membrane 5 and the outer stiffening tube part 9c.

The inner and the outer tube membrane 4, 5 are manufactured of a flexible and expandable material. Therefore, when an overpressure is generated in the pressure channel 3 by feeding the pressure medium 8, that is, in the shown pressure-loaded tube condition, by the effect of a radially outward-pointing pressure force component Fa, the outer tube membrane 5 presses the adjacent inner stiffening tube part 10a radially toward the outside while expanding. As a result, its wire mesh is firmly pressed under pressure against the wire mesh interior side of the outer stiffening tube part 9c so that a frictionally engaged/form-locking connection is established between the two coaxial tube parts 10a and 9c, whereby the flexurally stiffer, stiffened state of the tube is caused. For this purpose, for example, an overpressure in the range of approximately 5 bar to approximately 40 bar can be used. In embodiments having several flexurally stiffer tube conditions, a selection can be made between these by overpressures of different intensities, in which case, also the frictional contact surface between the flexible stiffening elements causing the stiffening is preferably appropriately coordinated with the providing of several flexurally stiffer conditions.

Specifically, the frictionally engaged connection is a result of the contact of the exterior side of the wire mesh of the inner stiffening tube part 10a with the interior side of the wire mesh of the outer stiffening tube part 9c. As a result of the frictionally engaged/form-locking connection, the inner stiffening tube part 10a stabilizes the outer stiffening tube part 9c in its longitudinal course also in possibly curved sections.

In addition, as a result of the overpressure in the pressure channel 3, the optional inner tube membrane 4 is pressed radially toward the interior against the usable-channel-forming inner element 1b with a pressure force component Fi pointing radially toward the interior, which is thereby placed closely against the latter from the outside. In this manner, the inner tube membrane 4 acts not only as a fluid-tight separation of the ring-shaped pressure channel 3 from the central working channel 2 but additionally protects the inner element 1b reliably from any radial expansion in cases in which, when the tube is in use, a certain overpressure occurs in the working channel 2, and supports it in its longitudinal course, also in curved sections; also see the above statements concerning the tube variations of FIGS. 1 to 9 which are equivalent to this extent.

When, starting from the stiffened tube condition, the pressure channel 3 is relieved again from the overpressure. That is, the pressure medium is drained from the pressure channel 3 until the pressure is equalized, the tube returns to its more flexible initial position, in which the optional inner tube membrane 4 loosely surrounds the inner element 1b, and the inner stiffening tube part 10a is situated without pressure in the annular gap between the outer tube membrane 5 and the outer stiffening tube part 9c.

It is understood that in further embodiments of the invention, arbitrary combinations of the components of the tube variations illustrated in FIGS. 1 to 11 are contemplated, particularly with respect to the flexible inner sleeve defining the usable channel and to the flexible outer sleeve.

Summarizing, the present invention therefore provides a tube that can be stiffened and can be changed in its stiffness condition in a controlled and defined manner between a state of lower bending stiffness and at least one state of an increased binding stiffness in that an overpressure is generated in a pressure channel which is provided separately from a usable channel. As a result of the overpressure, flexible stiffening elements are pressed against one another in a tube-shape-stiffening manner, for example by a frictional engagement and/or a form locking. The flexible stiffening elements comprise, for example, a flexible, not significantly expandable outer element, such as an outer sleeve in the form of a tube spring or a tubular woven texture jacket, and one or more inner members which can be pressed against the latter, for example, in the form of axially extending longitudinal wires or longitudinal rods. These can absorb tensile forces and, by way of a frictional engagement and/or form locking, stabilize the possibly curved longitudinal course of the outer element and thus the tube as a whole. For example, flat wires or round wires, strands or ropes can be used as inner members. As an alternative, an individual inner member in the form of a tube braiding or the like can be used that is radially expandable to the required extent. Instead of the illustrated linearly axial course, the inner members, as an alternative, may also, for example, be arranged with a helical or other course with an axial direction component.

Additional alternative embodiments of the invention can include the flexible inner members, like the illustrated longitudinal rods 10, being prefabricated on the exterior side of the outer tube membrane or on the interior side of the flexible outer sleeve, i.e., they are mounted thereon or constructed as an integrated part thereof in a manner not illustrated. When several inner members are present, it is also contemplated to implement some of them on the exterior side of the outer tube membrane and the remainder of them on the interior side of the flexible outer sleeve in a prefabricated manner.

1-10. (canceled)

11. A tube that can be controllably stiffened, comprising:
a pressure channel for controllable generation of a stiffening overpressure by introducing therein a pressure medium,
a plurality of flexible stiffening elements of which at least one is stable with respect to tensile force and which are pressed onto one another when acted upon by the stiffening overpressure to stiffen the tube from a more flexible to a flexurally stiffer state, and
a channel which is separated from the pressure channel and which, in the stiffer state of the tube, forms a working channel corresponding to a shape of the tube.

12. The tube according to claim 11, wherein the flexible stiffening elements are constructed for at least one of frictionally engagement and form-locking stiffening interaction, and comprise at least one of a flexible sleeve and at least one flexible longitudinal rod-type element and a flexible outer sleeve configured as an outer stiffening tube part and an inner stiffening tube part surrounded by the latter, which inner stiffening tube part, when acted upon by the stiffening overpressure, is configured to be pressed in a manner stiffening the tube shape against the inner side of the outer stiffening tube part.

13. The tube according to claim 11, wherein the pressure channel is constructed as a ring channel that is bounded by an inner and an outer tube membrane, of which at least the outer tube membrane is flexible and radially expandable to exert a radially outwardly-directed pressure force component as a result of the stiffening overpressure on the flexible stiffening elements which adjoin the outer tube membrane radially toward the outside.

14. The tube according to claim 13, wherein the flexible stiffening elements are constructed for at least one of frictionally engagement and form-locking stiffening interaction, and comprise at least one of a flexible sleeve and at least one flexible longitudinal rod-type element and a flexible outer sleeve configured as an outer stiffening tube part and an inner stiffening tube part surrounded by the latter, which inner stiffening tube part, when acted upon by the stiffening overpressure, is configured to be pressed in a manner stiffening the tube shape against the inner side of the outer stiffening tube part.

15. The tube according to claim 13, wherein the working channel is bounded by a flexible inner sleeve that is surrounded by the inner tube membrane or simultaneously forms the inner tube membrane.

16. The tube according to claim 13, wherein the flexible stiffening elements comprise a flexible outer sleeve and at least one inner member arranged between the outer tube membrane and the outer sleeve, which inner member by the admission of pressure is arranged to be pressed by the radially outwardly-directed pressure force component radially toward the outside against the flexible outer sleeve.

17. The tube according to claim 15, wherein the working channel is bounded by a flexible inner sleeve that is surrounded by the inner tube membrane or simultaneously forms the inner tube membrane.

18. The tube according to claim 15, wherein at least one of the flexible outer sleeve and the flexible inner sleeve is formed by a tube jacket with a cast-in helical spring or a cast-in tubular woven fabric or by a helical spring.

19. The tube according to claim 15, wherein the at least one inner member is prefabricated together with the outer tube membrane on its exterior side or together with the outer sleeve on its interior side.

20. The tube according to claim 19, wherein at least one of the flexible outer sleeve and the flexible inner sleeve is formed by a tube jacket with a cast-in helical spring or a cast-in tubular woven fabric or by a helical spring.

21. The tube according to claim 12, wherein a plurality of longitudinal rod-type elements or inner members extending with an axial direction component are distributed circumferentially.

22. The tube according to claim 21, wherein the pressure channel is constructed as a ring channel that is bounded by an inner and an outer tube membrane, of which at least the outer tube membrane is flexible and radially expandable to exert a radially outwardly-directed pressure force component as a result of the stiffening overpressure on the flexible stiffening elements which adjoin the outer tube membrane radially toward the outside.

23. The tube according to claim 12, wherein the at least one longitudinal rod-type element or inner member is constructed from one of flat wire material, round wire material, strand material and rope material, or by a tube braiding.

24. The tube according to claim 23, wherein the pressure channel is constructed as a ring channel that is bounded by an inner and an outer tube membrane, of which at least the outer tube membrane is flexible and radially expandable to exert a radially outwardly-directed pressure force component as a result of the stiffening overpressure on the flexible stiffening elements which adjoin the outer tube membrane radially toward the outside.

25. The tube according to claim 13, wherein the inner stiffening tube membrane is prefabricated together with the outer tube membrane on an exterior side thereof or together with the outer stiffening tube part on an interior side thereof.
